(19) United States
(12) Patent Application Publication
(10) Pub. No.: US 2019/0100802 A1
(43) Pub. Date: Apr. 4, 2019

(54) MARKER FOR PREDICTING PRESSURE ULCER DEVELOPMENT AND USE THEREOF

(71) Applicant: THE UNIVERSITY OF TOKYO, Tokyo (JP)

(72) Inventors: Hiromi SANADA, (US); Gojiro NAKAGAMI, (US); Takeo MINEMATSU, (US)

(73) Assignee: THE UNIVERSITY OF TOKYO, Tokyo (JP)

(21) Appl. No.: 16/068,498

(22) PCT Filed: Jan. 13, 2017
(86) PCT No.: PCT/JP2017/001020

§ 371 Date: Dec. 12, 2018

Related U.S. Application Data
(60) Provisional application No. 62/278,454, filed on Jan. 14, 2016.

(51) Int. Cl. C12Q 1/6881; A01K 67/027; C12Q 1/686

(52) U.S. Cl. CPC C12Q 1/6881; A01K 67/0273; C12Q 1/686; A01K 2227/105; C12Q 2600/106; C12Q 2600/158; C12Q 2600/118; C12Q 2600/112; A01K 2267/0368

(57) ABSTRACT

A marker for predicting pressure ulcer development, selected from the group consisting of interleukin (IL)-1α, vascular endothelial growth factor (VEGF)-C, plasminogen activator inhibitor (PAI)-1, and heat-shock protein (HSP) 90α.

### TECHNICAL FIELD

The present invention relates to a marker for predicting pressure ulcer development and use thereof. More specifically, the present invention relates to a marker for predicting pressure ulcer development, a kit for predicting pressure ulcer development, a method for predicting pressure ulcer development, and a method for producing a pressure ulcer non-human animal model. Priority is claimed on U.S. 62/278,454, provisionally filed in the United States on Jan. 14, 2016, the content of which is incorporated herein by reference.

### BACKGROUND ART

A pressure ulcer is a life-threatening disease of particular concern among elderly patients, but the occurrence of the disease cannot be completely prevented (for example, refer to NPL 1).

Pressure ulcers are formed by four pathways: ischemic disorder, reperfusion injury, lymphatic dysfunction, and mechanical deformation. The severity of a pressure ulcer is generally classified by the “depth (invasion depth)”. A representative classification system is the method proposed by the National Pressure Ulcer Advisory Panel (NPUAP) of the United States. According to the classification method, the severity of a pressure ulcer is classified into Category I (non-blanchable erythema), Category II (partial thickness loss of dermis), Category III (full-thickness tissue loss), and Category IV (full-thickness tissue loss with exposure of bone, tendon, or muscle).

### CITATION LIST

### Non-Patent Literature

[NPL 1] Allman, R. M., Pressure ulcers among the elderly, N. Engl. J. Med., 320 (13), 850-853, 1989.

### SUMMARY OF INVENTION

### Technical Problem

It is considered that if pressure ulcer development can be predicted, it is possible to prevent pressure ulcer development by performing appropriate intervention. In order to predict pressure ulcer development, it is necessary to directly detect a response of a tissue to pressure loading. However, currently, an effective marker capable of predicting pressure ulcer development is not known. With this, an object of the present invention is to provide a technique of predicting pressure ulcer development.

### Solution to Problem

The present invention includes the following aspects.

[1] A marker for predicting pressure ulcer development, selected from the group consisting of interleukin (IL)-1α, vascular endothelial growth factor (VEGF)-C, plasminogen activator inhibitor (PAI)-1, and heat-shock protein (HSP) 90α.

[2] A kit for predicting pressure ulcer development, including a primer set for amplifying cDNA of IL-1α, PAI-1, or HSP90α, a probe specifically hybridizes with mRNA of VEGF-C, PAI-1, or HSP90α, and a specific binding substance to a IL-1α protein, a VEGF-C protein, a PAI-1 protein, or an HSP90α protein.

[3] A method for predicting pressure ulcer development, including measuring the expression amount of IL-1α, VEGF-C, PAI-1, or HSP90α in a specimen derived from a subject area of a patient.

[4] The method for predicting pressure ulcer development according to [3], in which the specimen is a membrane specimen obtained by attaching a polar membrane wetted with water, a physiological saline solution, or a buffer solution, to a subject area of the skin of a patient, leaving the resultant for a predetermined time, and then recovering thereof, and measuring the expression amount of IL-1α, VEGF-C, PAI-1, or HSP90α is carried out by measuring an existing amount of a IL-1α protein, a VEGF-C protein, a PAI-1 protein, or an HSP90α protein in the membrane specimen.

[5] The method for predicting pressure ulcer development according to [3] or [4], further including predicting pressure ulcer development in the subject area, in a case where the expression amount of IL-1α, VEGF-C, or PAI-1 is increased compared to that of a control.

[6] The method for predicting pressure ulcer development according to [3] or [4], further including predicting pressure ulcer development in a subject area, in a case where redness is observed in the subject area and the expression amount of HSP90α is equivalent to that of a control.

[7] A method for predicting pressure ulcer development, including measuring an existing amount of a PAI-1 protein in a blood specimen derived from a patient and predicting pressure ulcer development in the patient in a case where the existing amount of the PAI-1 protein is increased compared to that of a control.

[8] A method for producing a pressure ulcer non-human animal model, including pinching the skin of a non-human animal and loading a pressure of 133.322 kPa for 6 hours.

[9] The method for producing a pressure ulcer non-human animal model according to [8], in which the non-human animal is a mouse and the skin is dorsal skin.

### Advantageous Effects of Invention

According to the present invention, it is possible to provide a technique of predicting pressure ulcer development.

### BRIEF DESCRIPTION OF DRAWINGS

FIG. 1 is a photograph showing an aspect of loading a pressure to a mouse in Experimental Example 1.

FIG. 2 is a representative photograph showing a result of observing the skin of a mouse of each group with the naked eye in Experimental Example 2.

FIGS. 3(a) to 3(f) are representative photographs showing histological analysis results of the skin tissue of a mouse of each group in Experimental Example 3.

FIG. 4 is a photograph showing a representative result of detecting the expression of HIF-1α protein by immunostaining in Experimental Example 4.

FIG. 5 is a photograph showing a representative result of detecting 8-OHdG by immunostaining in Experimental Example 4.

FIG. 6 is a photograph showing a representative result of detecting LYVE-1 protein by immunostaining in Experimental Example 4.

FIG. 7 is a photograph showing a representative result of staining a tissue section with hematoxylin and eosin in Experimental Example 4.

FIG. 8 is a photograph showing a representative result of detecting the expression of PAI-1 protein in Experimental Example 5.

FIG. 9 is a photograph showing a representative result of detecting the expression of IL-1α protein in Experimental Example 5.

FIG. 10 is a photograph showing a representative result of detecting the expression of VEGF-C protein in Experimental Example 5.

FIG. 11 is a photograph showing a representative result of detecting the expression of HSP90α protein in Experimental Example 5.

FIG. 12 is a graph showing a result of examining the expression of PAI-1 protein in Experimental Example 6.

FIG. 13 is a graph showing a result of examining the expression of IL-1α protein in Experimental Example 6.

FIG. 14 is a graph showing a result of examining the expression of VEGF-C protein in Experimental Example 6.

FIG. 15 is a graph showing a result of examining the expression of HSP90α protein in Experimental Example 6.

### DESCRIPTION OF EMBODIMENTS

[Marker for Predicting Pressure Ulcer Development]

In Embodiment 1, the present invention provides a marker for predicting pressure ulcer development, selected from the group consisting of VEGF-C, PAI-1, and HSP90α.

As described in examples, the present inventors clarified that an increase in the expression amount of IL-1 cc, VEGF-C, and PAI-1 is associated with pressure ulcer development. In addition, the present inventors clarified that it is possible to predict pressure ulcer development in a case where the expression amount of HSP90α is equivalent to that of a control, and redness of the skin is observed.

Therefore, IL-1α, VEGF-C, PAI-1, or HSP90α, can be a marker for predicting pressure ulcer development. The marker for predicting pressure ulcer development of the present embodiment may be detected at a protein level, or may be detected at a gene level.

With the marker for predicting pressure ulcer development of the present embodiment, it is possible to accurately predict pressure ulcer development, which is difficult to predict in the related art.

In the examples below, it is possible to predict pressure ulcer development noninvasively by detecting the marker for predicting pressure ulcer development at a protein level by skin blotting.

Skin blotting is a method of attaching a membrane such as a nitrocellulose membrane or a PVDF membrane to the skin for a predetermined time and recovering thereof, and analyzing a protein captured by the recovered membrane using immunostaining. The predetermined time is not particularly limited. For example, the predetermined time may be 1 minute to 24 hours. According to skin blotting, it is possible to detect the expression and secretion of a protein noninvasively.

[Kit for Predicting Pressure Ulcer Development]

In Embodiment 1, the present invention provides a kit for predicting pressure ulcer development, including a primer set for amplifying cDNA of IL-1α, VEGF-C, PAI-1, or HSP90α, a probe specifically hybridize with mRNA of IL-1α, VEGF-C, PAI-1, or HSP90α, and a specific binding substance to an IL-1α protein, a VEGF-C protein, a PAI-1 protein, or an HSP90α protein.

With the kit of the present embodiment, it is possible to detect the expression of IL-1α, VEGF-C, PAI-1, or HSP90α at a protein level or at a gene level, and to predict pressure ulcer development.

(Primer Set)

The primer set is not particularly limited as long as cDNA of genes of IL-1α, VEGF-C, PAI-1, HSP90α can be amplified. RefSeq ID of human IL-1α genes is NM_000575, and RefSeq ID of mouse IL-1α genes is NM_010554. In addition, RefSeq ID of human VEGF-C genes is NM 005429, and RefSeq ID of mouse VEGF-C genes is NM_009506. In addition, RefSeq ID of human PAM genes is NM_000602, and RefSeq ID of mouse PAI-1 genes is NM 008871. In addition, RefSeq ID of human HSP90α genes is NM_001017963, and RefSeq ID of mouse HSP90α genes is NM_010480.

(Probe)

The probe is not particularly limited as long as the probe can be specifically hybridized into mRNA of genes of IL-1α, VEGF-C, PAI-1, or HSP90α. The probe may be fixed on an antibody and configure a DNA microarray and the like.

(Specific Binding Substance)

Examples of the specific binding substance include an antibody, antibody debris, an aptamer, and the like. The antibody may be prepared by immunizing an animal such as a mouse with a subject protein or a partial peptide thereof as an antigen. Or, an antibody can be prepared by screening and the like of an antibody library such as phage library. In addition, examples of the antibody debris include by, Fab, scFv, and the like. The antibody or the antibody debris may be polyclonal or monoclonal.

The aptamer is a substance having a specific binding capacity to a marker. Examples of the aptamer include a nucleic acid aptamer, a peptide aptamer, and the like. The nucleic acid aptamer having a specific binding capacity to a subject protein can be selected by the systematic evolution of ligand by exponential enrichment (SELEX) method and the like, for example. In addition, the peptide aptamer having a specific binding capacity to a subject protein can be selected by the two-hybrid method using yeast and the like.

The specific binding substance is not particularly limited as long as the specific binding substance can specifically bind to a subject protein, and may be a commercially available substance. In addition, the specific binding substance may configure a protein chip fixed on an antibody and the like.

[Method for Predicting Pressure Ulcer Development]

In Embodiment 1, the present invention provides a method for predicting pressure ulcer development including measuring the expression amount of IL-1α, VEGF-C, PAI-1, or HSP90α in a specimen derived from a subject area of a patient.

In the method for predicting pressure ulcer development of the present embodiment, examples of the patient include a patient with suspected occurrence of a pressure ulcer. In addition, examples of the subject area include an area in which occurrence of a pressure ulcer is suspected. In addition, examples of the specimen include a biopsy tissue, blood, a specimen recovered by skin blotting, and the like.

Measurement of the expression amount of IL-1α, VEGF-C, PAI-1, or HSP90α may be performed at an mRNA level, or may be performed at a protein level.

In a case where the expression amount of IL-1α, VEGF-C, PAI-1, or HSP90α is detected at an mRNA level, for example, it is possible to detect the expression of the marker genes by performing RT-PCR using the entire RNA or mRNA extracted from a biopsy specimen, a biological specimen such as blood, and the like as a sample. Detection of the expression of genes may be performed by qualitative PCR, for example, may be performed by a quantitative gene amplification method such as a real time quantitative PCR and the like, for example, and may be performed by a gene amplification method in which reaction proceeds at a constant temperature such as LAMP method. Or, the expression of marker genes may be detected by using DNA microarray and the like, for example.

In a case where the expression of marker genes is detected at a protein level, for example, presence of a marker protein may be detected by immunostaining, immunoblotting, ELISA, and the like using a biopsy tissue, blood, a specimen recovered by skin blotting, and the like as a sample.

In the method for predicting pressure ulcer development of the present embodiment, the specimen may be a membrane specimen obtained by attaching a polar membrane wetted with water, a physiological saline solution, or a buffer solution, to a subject area of the skin of a patient, leaving the resultant for a predetermined time, and then recovering thereof, and measuring the expression amount of IL a, VEGF-C, PAI-1, or HSP90α may be carried out by measuring an existing amount of a IL-1α protein, a VEGF-C protein, a PAI-1 protein, or an HSP90α protein in the membrane specimen.

That is, the method for predicting pressure ulcer development of the present embodiment can be performed by skin blotting. With this, it is possible to predict pressure ulcer development noninvasively.

Here, examples of the membrane with polarity include a nitrocellulose membrane, a PVDF membrane, and the like. In addition, the predetermined time is not particularly limited, and the example of the predetermined time includes 1 minute to 24 hours.

In the method for predicting pressure ulcer development of the present embodiment, in a case where the expression amount of IL-1α, VEGF-C, or PAI-1 is increased compared to that of a control, it may be predicted that a pressure ulcer has developed in the subject area. Here, as the control, a specimen collected from an area where a pressure ulcer is apparently not developed derived from the same patient, a specimen derived from a healthy person not having a pressure ulcer, and the like can be used.

Or, in the method for predicting pressure ulcer development of the present embodiment, in a case where redness is observed in the subject area of the patient and the expression amount of HSP90α, is equivalent to that of a control, it may be predicted that a pressure ulcer has developed in the subject area. Here, as the control, a specimen collected from the area in which a pressure ulcer does not clearly occur, derived from the same patient, specimen derived from a healthy person not having a pressure ulcer, and the like can be used.

Or, the method for predicting pressure ulcer development of the present embodiment may include measuring an existing amount of a PAI-1 protein in a blood specimen derived from a patient, and predicting pressure ulcer development of the patient in a case where the existing amount of the PAI-1 protein is increased compared to that of a control. Here, as the control, a blood specimen derived from the same patient before pressure ulcer development, a blood specimen derived from a healthy person not having a pressure ulcer, and the like can be used.

Since the PAI-1 protein secreted from a tissue easily flows into blood, it is possible to predict pressure ulcer development by measuring the PAI-1 protein in the blood.

[Method for Producing Pressure Ulcer Non-Human Animal Model]

In Embodiment 1, the present invention provides a method for producing a pressure ulcer non-human animal model, including pinching the skin of a non-human animal and loading a pressure of 133.322 kPa for 6 hours. The non-human animal is not particularly limited, and examples of the non-human animal include cats, dogs, horses, monkeys, cows, sheep, pigs, goats, rabbits, hamsters, guinea pigs, rats, and mice.

In the production method of the present embodiment, for example, the non-human animal may be a mouse, and the skin to which a pressure is loaded may be dorsal skin.

In examples described later, it was confirmed that the pressure ulcer non-human animal model obtained by the production method of the present embodiment was acceptable as a Category I pressure ulcer animal model, as a result of observation of the skin by the naked eye, histological analysis, and immunohistochemical analysis. Since a pressure ulcer non-human animal model can be easily produced, a pressure ulcer non-human animal model can be used in the analysis of a pressure ulcer development mechanism and development of a prevention method or a treatment method of a pressure ulcer.

### EXAMPLES

The present invention will be described below using examples, but the present invention is not limited to the following examples.

### Experimental Example 1

(Production of pressure ulcer animal model)

Using a 9-week-old ICR mouse (SLC Japan), a Category I pressure ulcer animal model was prepared. The experiment was performed in accordance with the protocol approved by Tokyo University Animal Experiment Committee.

First, the mouse was fed and housed for one week. Subsequently, dorsal hair was removed using hair removal cream under anesthesia. A growth phase of hair in an area where hair follicles grow below the skin was induced. In the growth phase of hair, proteins secreted from a deeper skin layer move to epidermis via a transfollicular route, and thus can be captured by skin blotting.

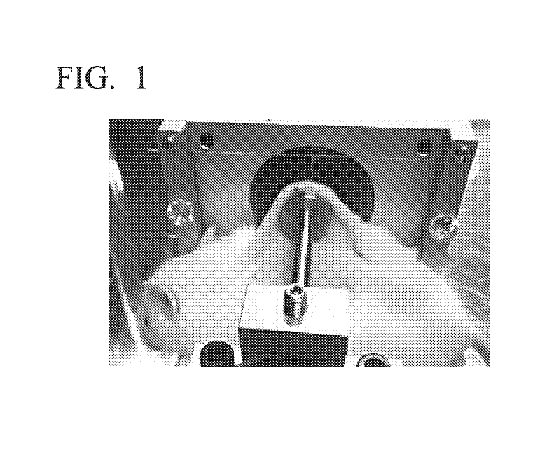

Subsequently, in order to eliminate the effects of inflammation caused by hair removal, the mouse was fed and recovered for one week until the start of experiment. Subsequently, the dorsal skin was shaved under anesthesia and a pressure of 133.322 kPa (1000 mmHg) was loaded using a pressure-loading device. FIG. 1 is a photograph showing an aspect in which a pressure is loaded.

A mouse group to which a pressure was loaded for 6 hours and a mouse group to which a pressure was loaded for 1 hour were prepared. In addition, a mouse to which a pressure was not loaded was set as a control group.

### Experimental Example 2

(Naked-Eye Evaluation of Pressure Ulcer Animal Model)

With respect to each mouse group, naked-eye evaluation on redness or purpura was performed before pressure loading to a mouse, immediately after, 30 minutes, 60 minutes, 90 minutes, 120 minutes, 24 hours, and 48 hours after pressure loading ended.

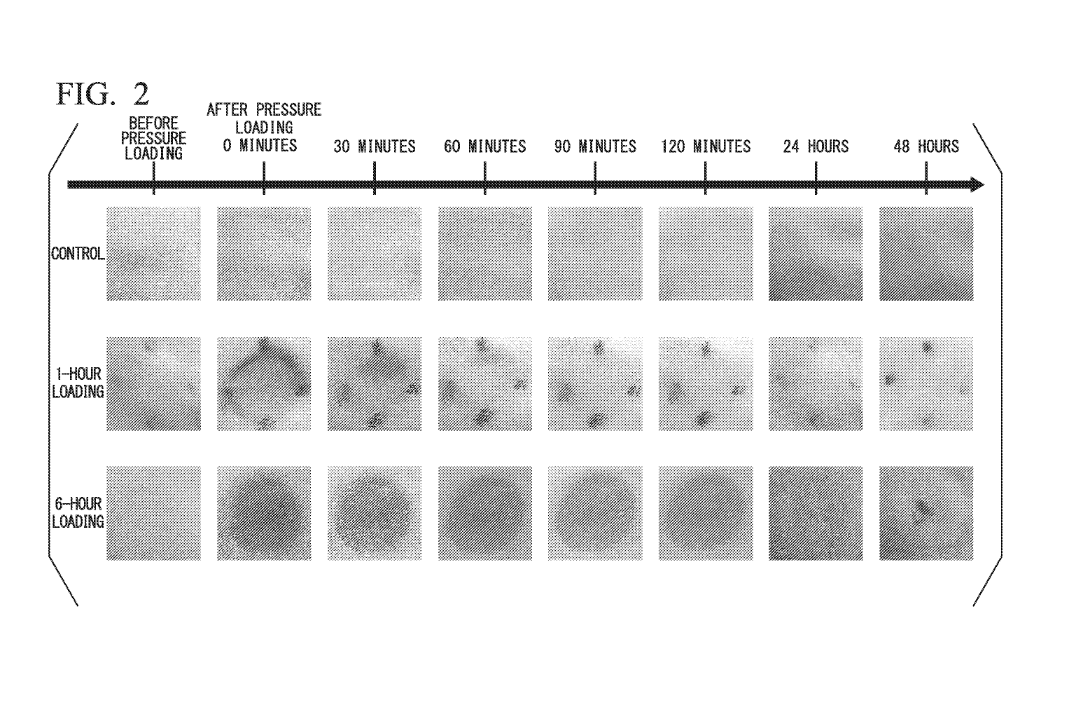

FIG. 2 is a representative photograph showing a result of naked-eye observation of the skin of each mouse group. The figure shows results before pressure loading to a mouse, immediately after, 30 minutes, 60 minutes, 90 minutes, 120 minutes, 24 hours, and 48 hours after pressure loading ended. The scale bar shows 5 mm. The mouse group to which a pressure was loaded for 6 hours was n=6, and the other groups were n=5.

In the mouse group to which a pressure was loaded for 1 hour, redness was observed immediately after pressure loading ended up to the 60-minute time point. On the other hand, in the mouse group to which a pressure was loaded for 6 hours, redness was observed immediately after pressure loading ended up to at least the 120-minute time point. Furthermore, pressure ulcer-like purpura was checked when pressure loading ended up to the 48-hour time point.

The above result shows that the mouse to which a pressure was loaded for 1 hour is acceptable as a blanchable erythema animal model. In addition, the result shows that the mouse to which a pressure was loaded for 6 hours is acceptable as a Category I pressure ulcer animal model.

### Experimental Example 3

(Histological Analysis of Pressure Ulcer Animal Model)

A skin tissue was collected 60 minutes and 48 hours after pressure loading to a mouse ended, and histological analysis was performed to evaluate tissue damage.

First, the collected skin tissue was fixed in a 4% paraformaldehyde solution at 4° C. for one night. Subsequently, dehydration was performed using ethanol and xylene, and paraffin was embedded. Subsequently, a tissue section having a thickness of 3.5 was prepared, and hematoxylin and eosin staining was performed.

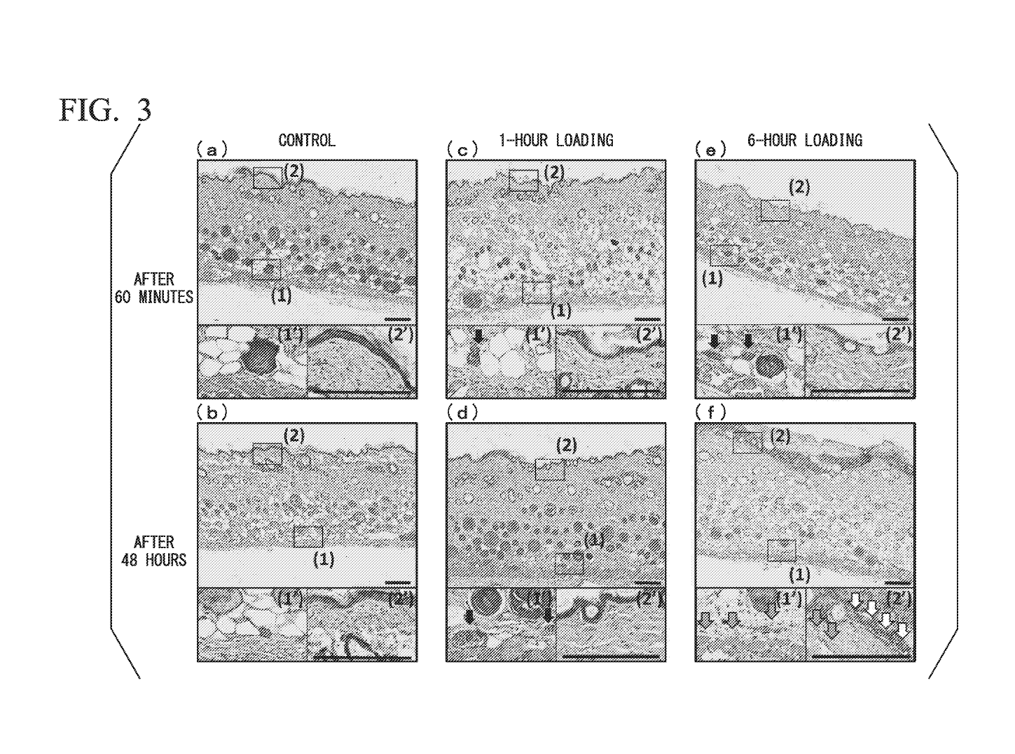

FIGS. 3(a) to 3(f) are representative photographs showing histological analysis result of skin tissues of each mouse group. FIGS. 3(a) and 3(b) are a result of a mouse of a control group. FIG. 3(a) shows a result of a skin tissue collected 60 minutes after pressure loading to mouse groups other than the control group ended. FIG. 3(b) shows a result of skin tissue collected 48 hours after pressure loading to mouse groups other than the control group ended. In FIGS. 3(a) and 3(h), an enlarged photograph of an area of (1) is indicated as (1′), and an enlarged photograph of an area of (2) is indicated as (2′). The scale shows 200 μm.

FIGS. 3(c) and 3(d) are results of mouse groups to which a pressure was loaded for 1 hour. FIG. 3(c) shows a result of a skin tissue collected 60 minutes after pressure loading to a mouse ended. FIG. 3(d) shows a result of a skin tissue collected 48 hours after pressure loading to a mouse ended. In FIGS. 3(c) and 3(d), an enlarged photograph of an area of (1) is indicated as (1′), and an enlarged photograph of an area of (2) is indicated as (2′). The scale shows 200 μm. The black arrows indicate thrombus-like aggregation of red blood cells.

FIGS. 3(e) and 3(f) are results of mouse groups to which a pressure was loaded for 6 hours. FIG. 3(e) shows a result of a skin tissue collected 60 minutes after pressure loading to a mouse ended. FIG. 3(f) shows a result of a skin tissue collected 48 hours after pressure loading to a mouse ended. In FIGS. 3(e) and 3(f), an enlarged photograph of an area (1) is indicated as (1′), and an enlarged photograph of an area of (2) is indicated as (2′). The scale shows 200 μm. The black arrows indicate agglutination of thromboid red blood cells. The white arrows indicate inflammatory cells infiltrating into dead cell debris due to inflammation. The gray arrows indicate bleeding.

As a result, as shown in FIGS. 3(a) and 3(b), the tissue section of the mouse of the control group showed a normal tissue structure. In addition, as shown in FIGS. 3(e) and 3(f), in the tissue section of the mouse group to which a pressure was loaded for 6 hours, there was checked frequent infiltration of inflammatory cells in addition to the inflammation checked in the tissue section of the mouse group to which a pressure was loaded for 1 hour. In addition, debris of dead cells due to inflammation and bleeding were checked 48 hours after pressure loading to a mouse ended.

The above result further supports that the mouse to which a pressure was loaded for 1 hour is acceptable as a blanchable erythema animal model, and the mouse to which a pressure was loaded for 6 hours is acceptable as a Category I pressure ulcer animal model.

### Experimental Example 4

(Immunohistochemical Analysis of Pressure Ulcer Animal Model)

In order to examine whether or not the prepared Category I pressure ulcer animal model has tissue damage caused by four pathways of ischemic disorder, reperfusion injury, lymphoid dysfunction, and mechanical deformation, associated with the formation of a pressure ulcer, immunohistochemical analysis was performed. As a specimen, a tissue section of a mouse of each group prepared as in Experimental Example 3 was used.

Specifically, in order to examine whether or not ischemic disorder occurred, a hypoxia inducible factor (HIF)-1α protein was detected. In addition, in order to examine whether or not reperfusion injury occurred, 8-hydroxy-2′-deoxyguanosine (8-OHdG) was detected. In addition, in order to examine whether or not lymphoid dysfunction occurred, lymphatic vessel endothelial hyaluronan receptor (LYVE)-1 protein was detected. In addition, mechanical deformation of cells was evaluated by the observation of morphological changes in fibroblasts.

In a case where ischemic disorder occurred in a pressure-loaded tissue, a low oxygen state was induced, expression of HIF-1α was increased, and nuclear translocation of the HIF-1α protein activated in accordance therewith occurred. Here, HIF-1α was used as a marker of ischemic disorder.

In addition, in the previous examination of the inventors, compared with a mouse in which an ischemic state was formed, in a mouse in which ischemia and reperfusion injury were formed, an increase in 8-OHdG was checked. Here, also in the present experimental example, 8-OHdG was used as an index indicating that reperfusion injury occurred.

In addition, in the previous examination of the inventors, in the skin of the mouse to which a pressure was loaded, reduction of LYVE-1 positive lymphatic ducts and lymphatic drainage disorder were checked. Here, also in the present experimental example, LYVE-1 protein was used as an index of lymphoid functional disorder.

In immunostaining other than 8-OHdG, endogenous peroxidase activities in a tissue section were quenched by leaving the tissue section at rest in a solution of a 0.3% hydrogen peroxide solution/a 20% methanol for 30 minutes.

In addition, in HIF-1α, staining, each tissue section was boiled in a 10 mM sodium citrate solution at 100° C. for 20 minutes to activate an antigen.

In LYVE-1 staining, it was necessary to boil the tissue section in a 10 mM sodium citrate solution added with 0.05% Tween20 (pH 6.0) at 100° C. for 20 minutes.

In addition, in 8-OHdG staining, it was necessary to autoclave the tissue section in a 10 mM sodium citrate solution (pH 6.0) at 121° C. for 15 minutes.

A primary antibody used in immunostaining was as follows. Anti-HIP-1α antibody (dilution 1:100, Novus Biologicals), anti-8-OHdG antibody (dilution 1:100, JaICA), and anti-LYVE-1-1 antibody (dilution 1:100, ReliaTech).

In addition, in HIF-1α and 8-OHdG staining, biotin-conjugated anti-rabbit IgG antibody (dilution 1:1000, Jackson Immuno Research) was used as secondary antibody.

In addition, in the LYVE-1 staining, peroxidase-conjugated anti-rabbit IgG antibody (dilution 1:1000, Jackson Immuno Research) was used as a secondary antibody.

<<Examination of Expression of HIF-1α Protein>>

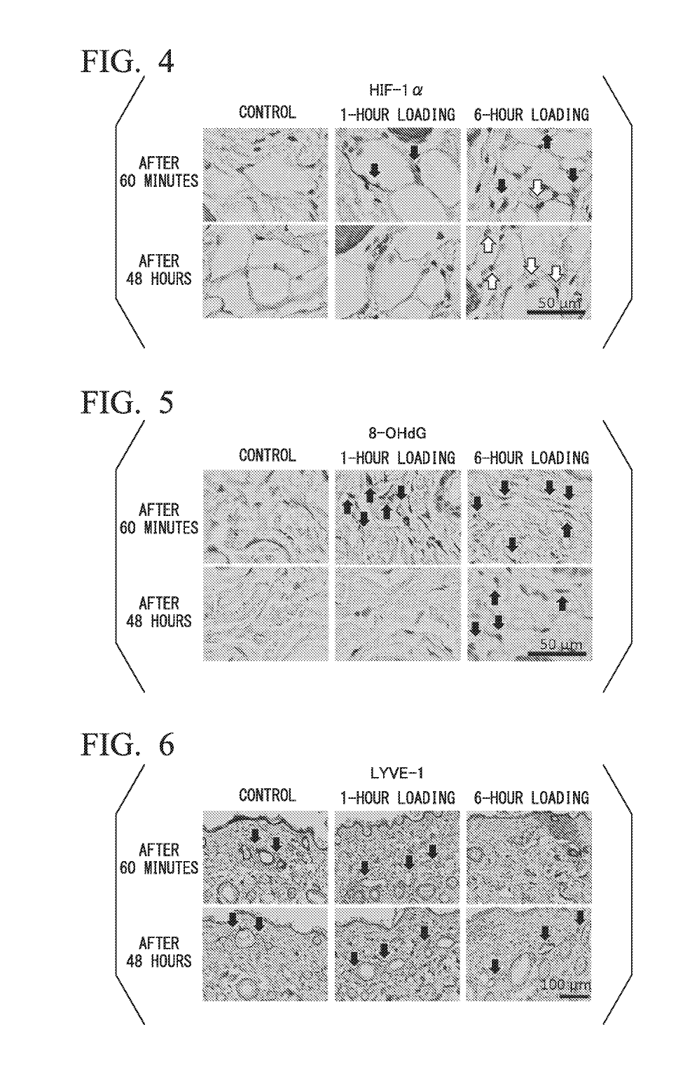

First, expression of HIF-1α protein was examined to evaluate ischemic disorder. FIG. 4 is a photograph showing a representative result of detecting the expression of HIF-1α protein by immunostaining. In FIG. 4, the black arrows indicate HIF-1 positive cells. In addition, the white arrows indicate nuclear translocation of the HIF-1α protein.

As a result, in the tissue of the mouse of the control group, HIF-1α positive cells were almost not observed. On the contrary, in the tissues of the mouse group to which a pressure was loaded for 1 hour and the mouse group to which a pressure was loaded for 6 hours, HIF-1α proteins were detected in cytoplasm of subcutaneous fat and muscular tissues 60 minutes after pressure loading to the mouse ended. In addition, in the tissue of the mouse group to which a pressure was loaded for 6 hours, nuclear translocation of the HIF-1α protein was observed 48 hours after pressure loading to the mouse ended.

<<Examination of Presence of 8-OHdG>>

Subsequently, presence of 8-OHdG was detected in order to evaluate reperfusion injury. FIG. 5 is a photograph showing a representative result of detecting 8-OHdG by immunostaining. In FIG. 5, the black arrows indicate 8-OHdG positive cells.

As a result, in the tissue of the mouse of the control group, 8-OHdG positive cells were almost not observed. On the contrary, in the tissues of the mouse group to which a pressure was loaded for 1 hour and the mouse group to which a pressure was loaded for 6 hours, fibroblast in which 8-OHdG were abundantly present was observed 60 minutes after pressure loading to the mouse ended.

In addition, in the tissue of the mouse group to which a pressure was loaded for 6 hours, 8-OHdG positive cells were observed in epidermal tissues and adipose tissues even 48 hours after pressure loading to the mouse ended.

<<Examination of Expression of LYVE-1 Protein>>

Subsequently, the LYVE-1 protein was stained in order to evaluate lymphoid dysfunction. FIG. 6 is a photograph showing a representative result of detecting the LYVE-1 protein by immunostaining. In FIG. 6, the black arrows indicate presence of the LYVE-1 protein.

As a result, in the tissue of the mouse of the control group, LYVE-1 positive ducts were observed in the upper dermis. On the contrary, in the tissue of the mouse group to which a pressure was loaded for 1 hour, expansion of lymphatic duct was frequently observed 60 minutes after pressure loading to the mouse ended. However, the lymphatic duct returned to the same level as that of the mouse of the control group 48 hours after pressure loading to the mouse ended.

On the other hand, in the tissue of the mouse group to which a pressure was loaded for 6 hours, LYVE-1 positive ducts were almost not observed 60 minutes after pressure loading to the mouse ended. However, expansion of the lymphatic duct was clearly observed 48 hours after pressure loading to the mouse ended.

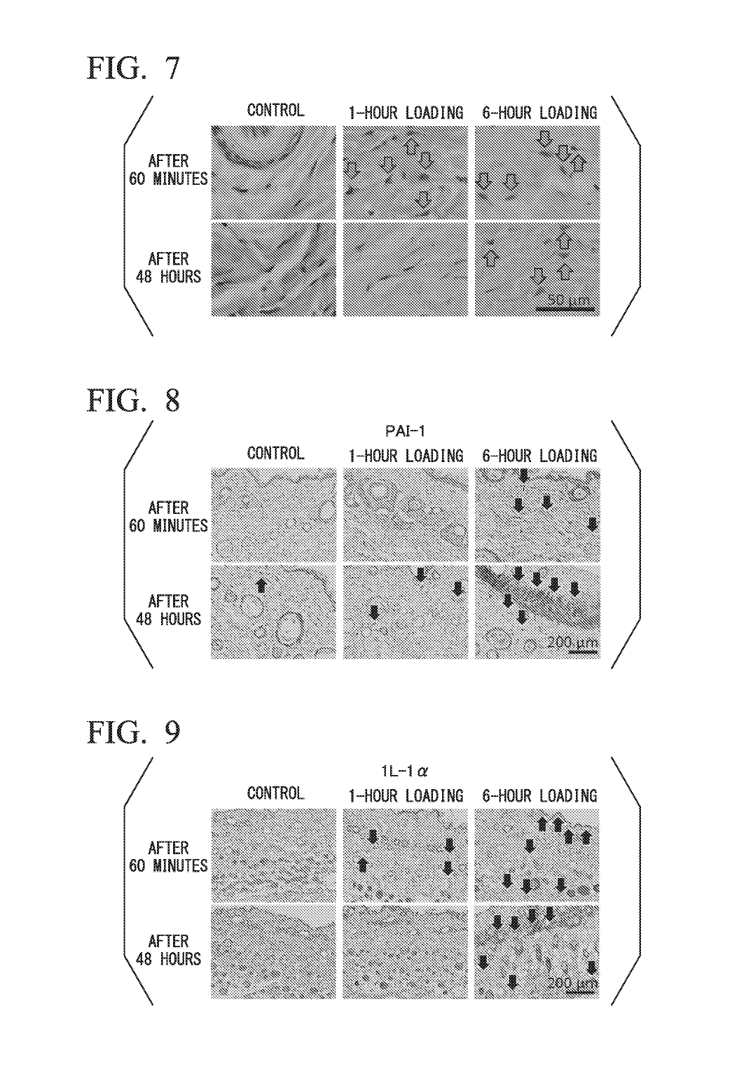

Subsequently, mechanical deformation of cells was evaluated by the observation of morphological changes in fibroblasts. FIG. 7 is a photograph showing a representative result of staining a tissue section with hematoxylin and eosin. In FIG. 7, the gray arrows indicate fibroblasts.

As a result, in the tissue of the mouse of the control group, almost all fibroblasts exhibited a typical spindle shape. On the contrary, in the tissue of the mouse group to which a pressure was loaded for 1 hour, fibroblast swelling was observed, and in the tissue of the mouse group to which a pressure was loaded for 6 hours, fibroblast swelling was observed in further higher frequency.

The above result further supports that the mouse to which a pressure was loaded for 1 hour is acceptable as a blanchable erythema animal model, and the mouse to which a pressure was loaded for 6 hours is acceptable as a Category I pressure ulcer animal model.

### Experimental Example 5

(Immunohistochemical Analysis of Expression of Marker for Predicting Pressure Ulcer Development in Pressure Ulcer Animal Model)

Subsequently, the inventors further examined expression of markers for predicting pressure ulcer development by immunostaining using a tissue section of the mouse of each group prepared as in Experimental Example 3. As the markers for predicting pressure ulcer development, expression of proteins of PAI-1, IL-1α, VEGF-C, and HSP90α was examined.

First, endogenous peroxidase activities in a tissue section were quenched by leaving each tissue section at rest in a solution of a 0.3% hydrogen peroxide solution/a 20% methanol for 30 minutes.

In the PAI-1 and IL-1α staining, each tissue section was boiled in a 10 mM sodium citrate solution at 100° C. for 20 minutes to activate an antigen.

In addition, in the VEGF-C and HSP90α staining, it was necessary to autoclave the tissue section in a 10 mM sodium citrate solution (pH 6.0) at 121° C. for 15 minutes.

A primary antibody used in immunostaining was as follows. Anti-PAI-1 antibody (dilution 1:100, Novus Biologicals), anti-IL-1α antibody (dilution 1:200, ProteinTech Group), and anti-VEGF-C antibody (dilution 1:100, Santa Cruz Biotechnology), and anti-HSP90α antibody (dilution 1:200, Lab Vision).

In addition, in the VEGF-C staining, biotin-conjugated anti-rabbit IgG antibody (dilution 1:1000, Jackson Immuno Research) was used as secondary antibody.

In addition, in the PAI-1, a, and HSP90α staining, peroxidase-conjugated anti-rabbit IgG antibody (dilution 1:1000, Jackson Immuno Research) was used as a secondary antibody.

<<Examination of Expression of PAI-1 Protein>>

FIG. 8 is a photograph showing a representative result of detecting the expression of PAI-1 protein. In FIG. 8, the black arrows indicate PAI-1 positive cells.

As a result, in the tissue of the mouse group to which a pressure was loaded for 6 hours, strong expression of PAI-1 protein was checked in the inflammatory cells infiltrating into dead cell debris due to inflammation 48 hours after pressure loading to the mouse ended.

The expression of PAL-1 protein was checked in various types of cells such as inflammatory cells, fibroblasts, vascular endothelial cells, and the like in tissue sections of the mouse group to which a pressure was loaded for 1 hour and the mouse group to which a pressure was loaded for 6 hours.

<<Examination of Expression of IL-1α Protein>>

FIG. 9 is a photograph showing a representative result of detecting the expression of IL-1α protein. In FIG. 9, the black arrows indicate IL-1α positive cells.

As a result, in the tissue of the mouse group to which a pressure was loaded for 6 hours, strong expression of the IL-1α protein was checked in the inflammatory cells infiltrating into dead cell debris due to inflammation 48 hours after pressure loading to the mouse ended.

The expression of IL-1α was checked in epidermal cells and vascular endothelial cells in the tissue section 60 minutes after pressure loading to the mouse ended, of the mouse group to which a pressure was loaded for 1 hour and the mouse group to which a pressure was loaded for 6 hours.

<<Examination of Expression of VEGF-C Protein>>

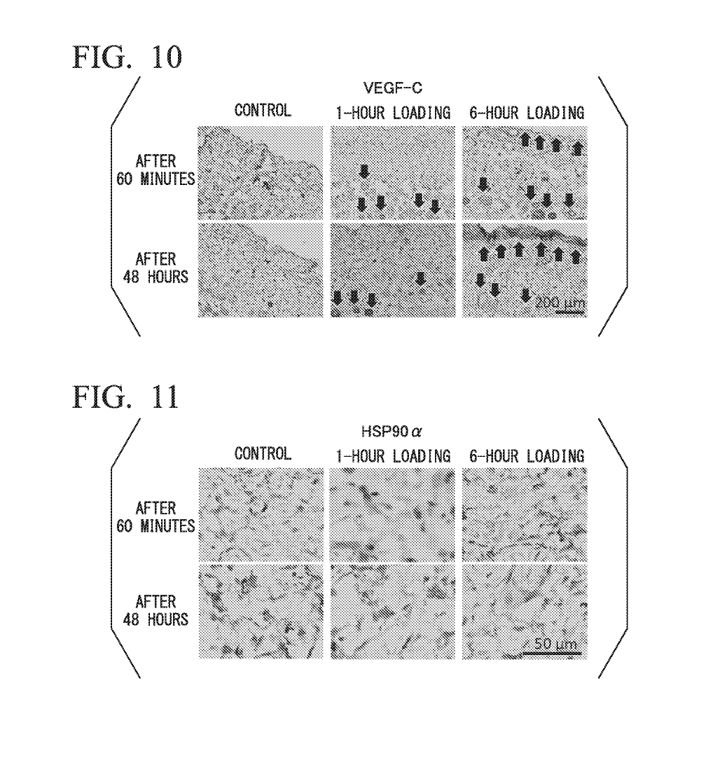

FIG. 10 is a photograph showing a representative result of detecting the expression of VEGF-C protein. In FIG. 10, the black arrows indicate VEGF-C positive cells.

As a result, in the tissue of the mouse group to which a pressure was loaded for 6 hours, strong expression of the VEGF-C protein was checked in epidermis and follicles in the inflammatory cells infiltrating into dead cell debris due to inflammation. In the tissue of the mouse group to which a pressure was loaded for 1 hour, the expression of the VEGF-C protein was checked only in the follicles.

<<Examination of Expression of HSP90α Protein>>

FIG. 11 is a photograph showing a representative result of detecting the expression of HSP90α protein. As a result, expression of the HSP90α protein was not checked in the tissue of any of the mouse groups.

From the above result, it is clearly possible to predict pressure ulcer development by detecting the expression of proteins of PAI-1, IL-1α, or VEGF-C.

### Experimental Example 6

(Analysis of Expression of Marker for Predicting Pressure Ulcer Development in Pressure Ulcer Animal Model by Skin Blotting)

Whether or not a marker for predicting pressure ulcer development can be detected noninvasively was examined by skin blotting. As the marker for predicting pressure ulcer development, expression of proteins of PAI-1, IL-1α, VEGF-C, and HSP90α was examined.

First, a nitrocellulose membrane (1×1 cm, Bio-Rad Laboratories) was immersed in 50 μL of physiological saline solution, and attached to an area of the skin of the mouse to which a pressure was loaded for 10 minutes. As a result, soluble proteins that leak from the epidermal tissue, the dermal tissue, and the subcutaneous tissue were captured by the nitrocellulose membrane via transepidermal and transfollicular routes. The recovered nitrocellulose membrane was held at 4° C. up to the analysis.

Subsequently, the nitrocellulose membrane was subjected to immunostaining. First, the nitrocellulose membrane was incubated in a solution of a 0.3% hydrogen peroxide solution/a 20% methanol solution to quench endogenous peroxidase activities. Subsequently, blocking was performed with a blocking solution (Type “Blocking One”, Nacalai Tesque).

Subsequently, each of the nitrocellulose membranes was divided into four pieces. Each of the pieces was stained with PAI-1 antibody (dilution 1:200, Novus Biologicals), anti-IL-1α antibody (dilution 1:200, ProtcinTech Group), anti-VEGF-C antibody (dilution 1:200, Santa Cruz Biotechnology), and anti-HSP90α antibody (dilution 1:200, Lab Vision), respectively.

As a secondary antibody, peroxidase-conjugated anti-rabbit IgG antibody (dilution 1:1000, Jackson Immuno Research) was used.

For detection, chemiluminescent substrates (Type “Luminata Forte”, Merck Milipore) were used, and a chemiluminescence detection device (Type “LumiCube”, Liponics) was used.

For analysis of skin blotting, a mean chemiluminescent signal intensity value was calculated over the entire membrane, with the exception of the edges of the nitrocellulose membrane. In addition, a relative value of chemiluminescent signal intensity (hereinafter, referred to as “relative mean signal intensity value” in cases) was obtained by dividing a test result by a result of a control mouse. Statistical analysis was performed by using Turkey's test. A value of p less than 0.05 was determined to be statistically significant.

<<Examination of Expression of PAI-1 Protein>>

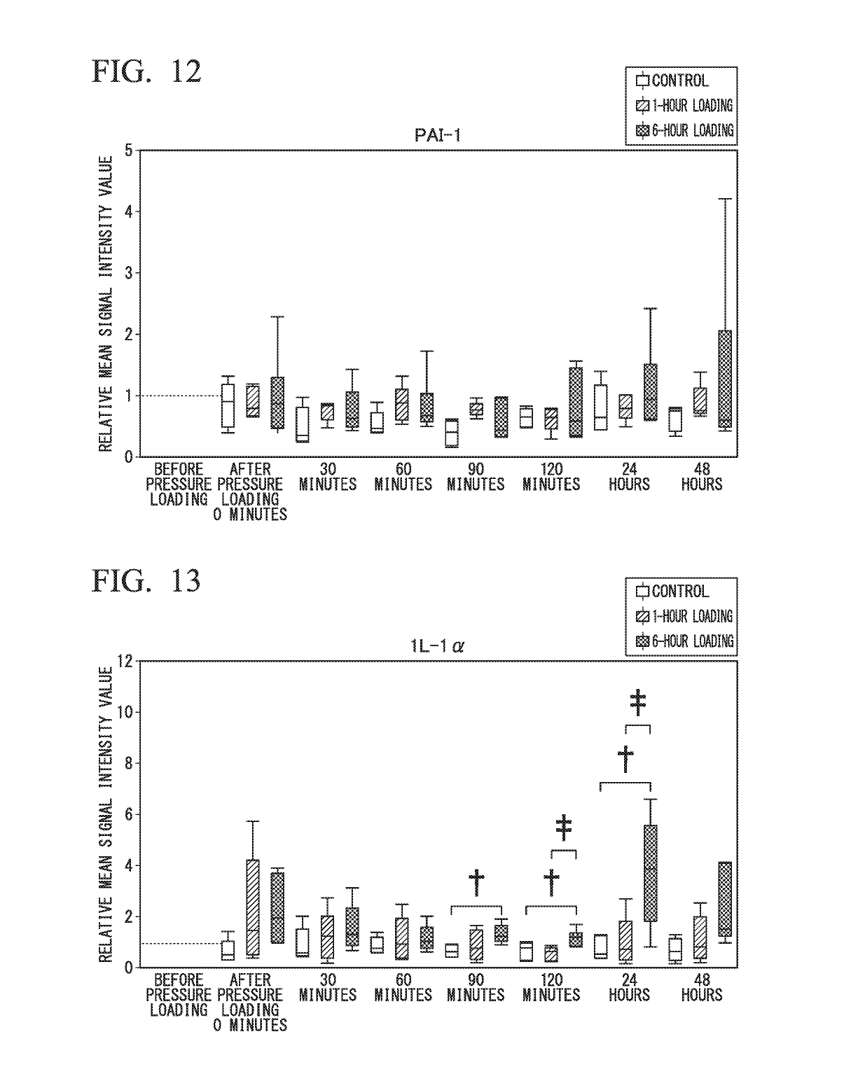

FIG. 12 is a graph showing a result of examining the expression of PAI-1 protein. As a result, expression of the PAI-1 protein did not show significant differences at any time point.

<<Examination of Expression of IL-1α Protein>>

FIG. 13 is a graph showing a result of examining the expression of IL-1α protein. In FIG. 13, “A” indicates that the value of p in the control group vs. the mouse group to which a pressure was loaded for 6 hours is less than 0.05, and “B” indicates that the value of p in the mouse group to which a pressure was loaded for 1 hour vs. the mouse group to which a pressure was loaded for 6 hours is less than 0.05.

As a result, it became clear that expression of the IL-1α protein was significantly higher compared with the control group in a specimen, 90 minutes, 120 minutes, and 24 hours after a pressure loading to the mouse ended, of the mouse group to which a pressure was loaded for 6 hours. The value of p was 0.046, 0.049, and 0.011, respectively.

Furthermore, it was clear that expression of the IL-1α protein was significantly higher compared with the mouse group to which a pressure was loaded for 6 hours in a specimen, 120 minutes and 24 hours after a pressure loading to the mouse ended, of the mouse group to which a pressure was loaded for 6 hours. The value of p was 0.016 and 0.018, respectively.

<<Examination of Expression of VEGF-C Protein>>

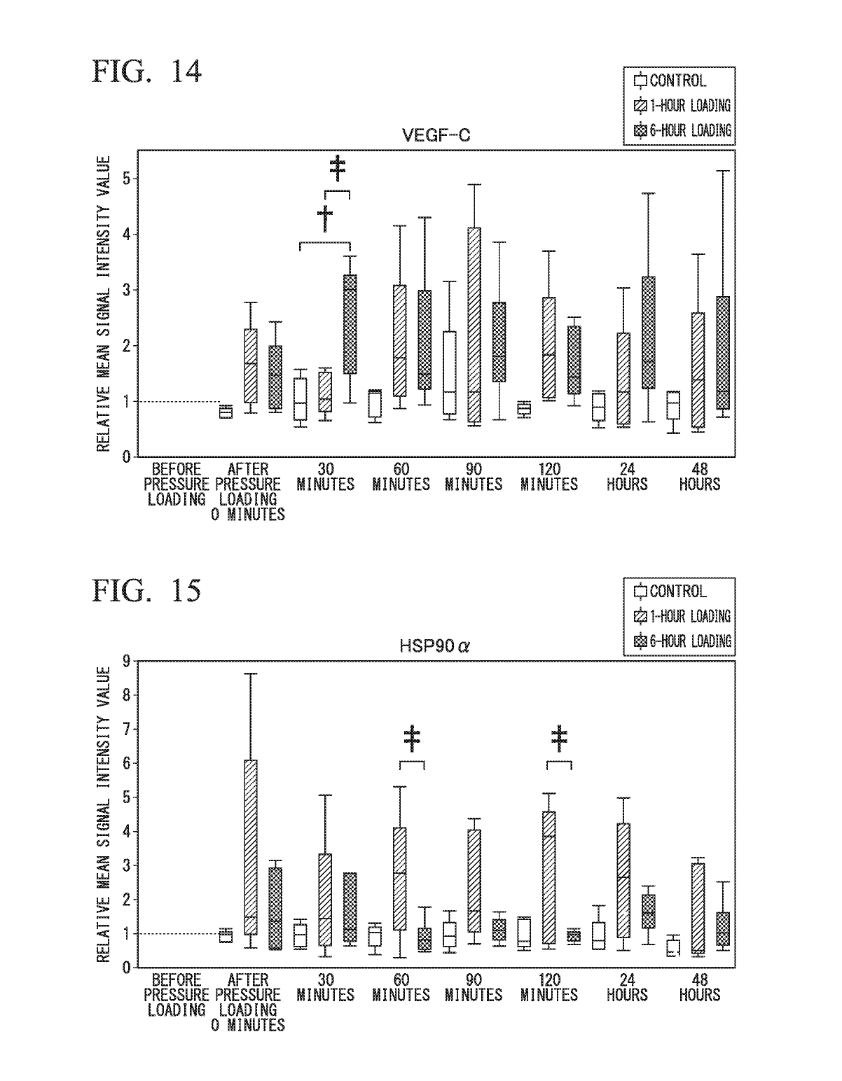

FIG. 14 is a graph showing a result of examining the expression of VEGF-C protein. In FIG. 14, “A” indicates that the value of p in the control group vs. the mouse group to which a pressure was loaded for 6 hours is less than 0.05, and “B” indicates that the p value in the mouse group to which a pressure was loaded for 1 hour vs. the mouse group to which a pressure was loaded for 6 hours is less than 0.05.

As a result, it became clear that expression of the VEGF-C protein was significantly higher compared with the control group and the mouse group to which a pressure was loaded for 1 hour in a specimen, 30 minutes after pressure loading to the mouse ended, of the mouse group to which a pressure was loaded for 6 hours. The value of p was 0.008 and 0.013, respectively.

<<Examination of Expression of HSP90α Protein>>

FIG. 15 is a graph showing a result of examining the expression of HSP90α protein. In FIG. 15, “A” indicates that the value of p in the mouse group to which a pressure was loaded for 1 hour vs. the mouse group is to which a pressure was loaded for 6 hours is less than 0.05.

As a result, it became clear that expression of the HSP90α protein was significantly higher compared with the mouse group to which a pressure was loaded for 6 hours in a specimen, 60 minutes and 120 minutes after pressure loading to the mouse ended, of the mouse group to which a pressure was loaded for 1 hour. The value of p was 0.047 and 0.041, respectively.

From the above results, it is clear that it is possible to predict pressure ulcer development noninvasively by detecting the expression of proteins of IL-1α, VEGF-C, or HSP90α by skin blotting.

### INDUSTRIAL APPLICABILITY

According to the present invention, it is possible to provide a technique of predicting pressure ulcer development.

1. (canceled)

2. A kit for predicting pressure ulcer development, comprising:
a primer set for amplifying cDNA of IL-1α, VEGF-C, PAI-1, or HSP90α;
a probe specifically hybridize with mRNA of IL-1α, VEGF-C, PAI-1, or HSP90α; and
a specific binding substance to a IL-1α protein, a VEGF-C protein, a PAI-1 protein, or an HSP90α protein.

3. A method for predicting and preventing pressure ulcer development, comprising:
measuring an expression amount of IL-1α, VEGF-C, PAI-1, or HSP90α in a specimen derived from a subject area of a patient,
predicting pressure ulcer development in the subject area, in a case where the expression amount of IL-1α, VEGF-C, or PAI-1 is increased compared to that of a control, and
performing appropriate intervention to prevent pressure ulcer development when pressure ulcer development is predicted.

4. The method for predicting and preventing pressure ulcer development according to claim 3,
wherein the specimen is a membrane specimen obtained by attaching a polar membrane wetted with water, a physiological saline solution, or a buffer solution, to a subject area of the skin of a patient, leaving the resultant for a predetermined time, and then recovering thereof, and
wherein the measuring the expression amount of IL-1α, VEGF-C, PAI-1, or HSP90α is carried out by measuring an existing amount of an IL-1α protein, a VEGF-C protein, a PAI-1 protein, or an HSP90α protein in the membrane specimen.

5. (canceled)

6. The method for predicting and preventing pressure ulcer development according to claim 3, further comprising:
predicting pressure ulcer development in a subject area, in a case where redness is observed in the subject area and the expression amount of HSP90α is equivalent to that of a control.

7. A method for predicting and preventing pressure ulcer development, comprising:
measuring an existing amount of a PAI-1 protein in a blood specimen derived from a patient,
predicting pressure ulcer development in the patient in a case where the existing amount of the PAI-1 protein is increased compared to that of a control, and
performing appropriate intervention to prevent pressure ulcer development when pressure ulcer development is predicted.

8. A method for producing a pressure ulcer non-human animal model, comprising:
pinching the skin of a non-human animal and loading a pressure of 133.322 kPa for 6 hours.

9. The method for producing a pressure ulcer non-human animal model according to claim 8,
wherein the non-human animal is a mouse and the skin is dorsal skin.
